(19) United States
(12) Patent Application Publication
(10) Pub. No.: US 2024/0165296 A1
(43) Pub. Date: May 23, 2024

(54) DRESSINGS AND METHODS FOR WOUND HEALING

(71) Applicant: The Curators of the University of Missouri, Columbia, MO (US)

(72) Inventors: Fateme FAYYAZBAKHSH, Rolla, MO (US); Ming C. LEU, Rolla, MO (US); Delbert E. DAY, Rolla, MO (US)

(73) Assignee: The Curators of the University of Missouri, Columbia, MO (US)

(21) Appl. No.: 18/281,704

(22) PCT Filed: Mar. 15, 2022
(86) PCT No.: PCT/US2022/020382

§ 371 Date: Sep. 12, 2023

Related U.S. Application Data
(60) Provisional application No. 63/161,389, filed on Mar. 15, 2021.

(51) Int. Cl. A61L 26/00; B33Y 10/00; B33Y 70/10; B33Y 80/00

(52) U.S. Cl. CPC A61L 26/0004; A61L 26/0066; A61L 26/008; B33Y 10/00; B33Y 70/10; B33Y 80/00; A61L 2300/102; A61L 2300/406; A61L 2300/64

(57) ABSTRACT

The current invention describes a method for developing bioactive borate glass (BBG)-hydrogel constructs based on 3D printing technology for healing of burn wounds and low-tomoderate exuding wounds. The hydrogels serve as a water reservoir and binder for BBG, to provide hydration of the BBG-hydrogel construct and to make the bioink printable, while 3D printing technology enables the layer-by-layer deposition of multiple materials including BG and hydrogels such as alginate, gelatin, GelMa, cellulose, chitosan and other like materials, as well as control of pore geometry to increase the available surface area for wound-dressing contact and more favorable cell-biomaterial interactions.

### FIELD OF THE INVENTION

The present invention relates to advanced wound care products. In particular, the present invention relates to dressings and associated methods for use of the dressings to treat wounds and burns, and to control bleeding.

### BACKGROUND OF THE INVENTION

Severe burn wounds are the most traumatic and physically debilitating injuries with local and systemic damages to the wound site. Early burn wound excision and autologous split-thickness skin grafts are the current gold standard in clinical practice that have significantly improved the outcomes for severely burned patients by reducing the mortality rate. However, the shortage in donor skin tissue, chronic graft rejection, impaired healing, infection, pain, and scarring are major challenges in burn wound treatment that have fostered the development of alternative approaches such as engineered tissues or synthetic skin substitutes. The majority of the wound healing products currently available do not fully recapitulate native skin, as they cannot replicate the layered structure of skin with regeneration of skin appendages. Due to the severe dehydration, electrolyte imbalance, and damage to the blood vessels, nerves, and underlying tissues, common treatments face many challenges in burn wound healing.

Biosynthetic wound dressings have exhibited positive clinical outcomes. However, these current commercial wound healing products cannot support the regeneration of skin appendages and scarless wound healing. Further, the healing processes associated with these wound healing products often results in a prolonged healing period that may be unacceptably long, as well as other healing conditions that may be less than ideal (e.g., exhibiting no autolytic debridement). Thus, there remains a need for new bioactive materials, wound dressings, and/or methods for preparing the bioactive materials and/or wound dressing products.

### BRIEF SUMMARY OF THE INVENTION

The present invention is directed to bioactive borate glass (BBG) wound dressings and processes for preparing the wound dressings. For example, the wound dressings of the present invention may be fabricated by 3D printing technology.

In one aspect, the present invention is directed to a wound dressing comprising a hydrogel matrix and up to 50 w/v % of a bioactive borate glass (BBG) comprising boron.

In another aspect, the present invention is directed to a process for preparing a bioactive borate glass (BBG) loaded hydrogel matrix. The process comprises providing a paste comprising hydrogels and BBG; additive manufacturing of the paste comprising hydrogels and BBG using extrusion-based 3D printing to form 3D printed constructs comprising the BBG and hydrogels; and crosslinking the 3D printed constructs to form a hydrogel matrix comprising up to about 50 w/v % BBG.

The invention is also directed to methods of treating wounds, burns, and/or controlling bleeding in a subject in need thereof. The method comprises applying the wound dressing to a wound, a burn, or a site of bleeding of the subject.

Other objects and features will be in part apparent and in part pointed out hereinafter.

### BRIEF DESCRIPTION OF THE SEVERAL VIEWS OF THE DRAWING

FIG. 1 illustrates different dressing shapes and sizes and pore geometries for 3D printing of porous BBG-hydrogel layers.

FIG. 2 illustrates an extrusion-based 3D printing technology apparatus comprising A) an extrusion-based bioprinter, B) multi-material parallel printing, and C) multi-material mixed printing.

FIG. 3 shows a wound dressing.

FIG. 4 shows a 3D printed BBG-hydrogel construct and the process equipment used to conduct the 3D printing.

FIG. 5 shows the rheological behavior of a BBG-hydrogel mixture vs. a hydrogel without BBG.

FIG. 6 shows the Young's modulus of a BBG-hydrogel mixture vs. a hydrogel without BBG.

FIG. 7 shows the results of a degradation test when a hydrogel was immersed in a phosphate buffered saline (PBS) solution.

FIG. 8 shows the total water content and hydration of BBG-hydrogel vs. hydrogel samples.

FIG. 9 shows the results of a cell viability test using MTT assay with human primary dermal fibroblasts.

FIG. 10 shows the results of in vivo wound healing tests in rat models.

FIG. 11 shows the appearance of wound healing in rat models.

FIG. 12 shows the H&E-stained slides for different treatment and control groups.

Corresponding reference characters indicate corresponding parts throughout the drawings.

### DETAILED DESCRIPTION OF THE INVENTION

Hydrogels are an essential class of polymers for dermal/epidermal regeneration due to their ability to donate or absorb water depending the wound condition. Wound care products often comprise biodegradable hydrogels to encourage wound healing within a moist environment. A wide variety of hydrogels exist, ranging from single-component hydrogels to complex and functionalized compositions reinforced with nanoparticles and growth factors.

In addition to the use of hydrogels, wound care products have progressed over time to include, for example, advanced skin substitutes containing cells and growth factors. Bioactive glasses are materials that have been shown to have considerable potential in biomedical applications. Bioactive glasses are amorphous solid structures that can be used as a substitute for hard tissue. Bioactive glasses are promising materials for soft tissue regeneration and wound healing applications. Silicate glass has been used in hard and soft tissue engineering. Non-silicate glass compositions for tissue regeneration have also been investigated. For example, biologically active elements such as B, Ag, Ca, Mg, Sr, Cu, and Zn can be incorporated into the glass networks. It is believed that bioactive glasses and corresponding dissolution products have a high potency for inducing anti-inflammatory response, angiogenesis, and antimicrobial activity.

Bioactive borate glass (BBG) is a type of bioactive glass comprising borate (B₂O₃) as the glass network matrix. Despite generally positive clinical results from utilizing BBG, in the form of a powder or fiber, on wet wounds (e.g., highly discharging wounds such as pressure ulcers), BBG by itself has been found to be unable to effectively treat dehydrated wounds (e.g. burn wounds and necrotic wounds).

Applicant has discovered that the 3D printed BBG-hydrogel mixture of the present invention increases the moisturizing activity of BBG and makes the mixture/matrix suitable for treating burn wounds. A printed BBG-hydrogel mixture has a porous dermal-like structure that is maintained in vivo with controlled vascularization and enhanced wound healing. Applying a 3D printed BBG-hydrogel mixture as a non-stick wound dressing also makes it easier to change the dressing with no pain or secondary trauma to the wound or surrounding tissue.

Recently, bioprinting (e.g., 3D printing) with bioinks consisting of different hydrogels and biocompatible polymers as well as living cells has been investigated to develop various tissue engineering constructs for hard and soft tissue regeneration. Skin bioprinting has also been investigated for artificial skins, synthetic grafts, and wound dressings using hydrogels. The use of 3D bioprinting allows for reproducible fabrication with bioinks, control over the printed structure, and an increased contact surface by adjusting the geometry.

In one embodiment, the present invention is directed to a wound dressing comprising a hydrogel matrix and up to 50 w/v % of a bioactive borate glass (BBG) comprising boron. For example, a wound dressing comprising a hydrogel matrix and 45 w/v % or less, 40 w/v % or less, 35 w/v % or less, 30 w/v % or less, 25 w/v % or less, 20 w/v % or less, 15 w/v % or less, 10 w/v % or less, or 5 w/v % or less of a bioactive borate glass (BBG) comprising boron.

One aspect of the present invention is directed to a method of 3D printing that incorporates BBG into a hydrogel matrix. The method comprises: providing a mixture of BBG-hydrogel paste in a controlled condition, additive manufacturing of the BBG-hydrogel paste using extrusion-based 3D printing, and chemical or physical crosslinking the 3D printed constructs. The 3D printed construction may be further sterilized as required.

In another embodiment, the present invention is directed to a process for preparing a bioactive borate glass (BBG) loaded hydrogel matrix. The process comprises providing a paste comprising hydrogels and BBG; additive manufacturing of the paste comprising hydrogels and BBG using extrusion-based 3D printing to form 3D printed constructs comprising the BBG and hydrogels; and crosslinking the 3D printed constructs to form a hydrogel matrix comprising up to about 50 w/v % a BBG. For example, 45 w/v % or less, 40 w/v % or less, 35 w/v % or less, 30 w/v % or less, 25 w/v % or less, 20 w/v % or less, 15 w/v % or less, 10 w/v % or less, or 5 w/v % or less of a BBG.

In another aspect, the present invention provides a method for applying the 3D printed dressings on different wound types including partial-thickness burn wounds, chronic wounds, etc. For example, the dressing may be applied to a wound, a burn, or a site of bleeding. In certain embodiments, the dressing is capable of autolytic debridement when applied to a wound, a burn, or a site of bleeding. In other embodiments, the dressing is capable of reducing or inhibiting scar tissue formation when applied to a wound, a burn, or a site of bleeding. In certain embodiment, the dressing is capable of inhibiting scar tissue formation when applied to a wound, a burn, or a site of bleeding.

In some embodiments, the wound dressing is capable of commercially acceptable moisture retention for up to 7 days after application to the wound, burn, or site of bleeding. For example, the wound dressing may be capable of commercially acceptable moisture retention for a period of at least about 7 days, at least about 8 days, at least about 9 days, at least about 10 days, at least about 11 days, at least about 12 days, at least about 13 days, at least about 14 days, or at least about 21 days.

In other embodiments, the wound dressing exhibits antibacterial activity for up to 7 days after application to the wound, burn, or site of bleeding. For example, the wound dressing exhibits antibacterial activity for a period of at least about 7 days, at least about 8 days, at least about 9 days, at least about 10 days, at least about 11 days, at least about 12 days, at least about 13 days, at least about 14 days, or at least about 21 days.

In a further aspect of the present invention, a method is provided for using the 3D printed BBG-containing dressing in combination with living cells for therapeutic tissue regeneration, development of tissue models for testing purposes, etc. The living cells are capable of migration, proliferation to the top and/or bottom, and survival within the dressing.

In certain embodiments, the present invention is directed to methods for preparing dressings for burn wounds and other dry wounds comprising BBG mixed with hydrogels. Still further, the dressing may be prepared using a 3D bioprinting technology. Without being bound by the theory, it is believed that the hydrogel acts as a water reservoir and a carrier for the BBG, which aids the printing process when utilizing a 3D bioprinting technology.

3D printed wound dressings of the present invention may comprise a matrix of hydrogels such as alginate, gelatin, GelMa, cellulose, chitosan and other like materials as well as their mixtures, combined with up to 50 w/v % BBG powder consisting of micrometer and/or nanometer sized particles. The mixture results in a BBG powder that is extrudable for 3D printing and a hydrogel that provides moisturizing activity. The ratio of BBG and hydrogel can be adjusted for printability and other functionalities such as degradability, hydration ability, cell viability, etc. The printed BBG-hydrogel mixture has a porous dermal-like structure that can be maintained in vivo for up to 7 days with controlled vascularization and enhanced wound healing.

3D printing technology enables the layer-by-layer deposition of BBG-hydrogel bioink, with numerous positive effects including: (i) allowing the dressings to be fabricated at any desired shapes and dimensions, conforming to the wound geometry if needed, (ii) including pores of any desired pore geometry to increase the available surface area for wound interaction, thus improving hydration and delivering the active ingredients in the BBG more effectively to the wound site for tissue regeneration, and (iii) enabling dressings to be made with multiple materials that vary between and within layers to improve the dressing's functions and performance.

In certain embodiments, a bioink may be formed from a paste made by combining BBG and a hydrogel matrix. The bioink may be used to print a component of a wound dressing. For example, in one embodiment, the bioink is printed by a process comprising additive manufacturing using extrusion-based 3D printing to form 3D printed constructs. The 3D printed constructs may then be crosslinked to form a hydrogel matrix. The hydrogel matrix may be used as a component of the wound dressing. It is believed, without being bound by the theory, that the bioink increases the hydration activity of BBG allowing the resulting dressing to provide a moist environment conducive for burn wound healing. Further, the BBG acts to support increased new tissue formation.

In still a further embodiment, the present invention is directed to a wound dressing as described or prepared above, wherein the BBG-hydrogel mixture of the wound dressing acts as a non-stick wound dressing to ensure that the dressing exchanges painlessly and without secondary trauma to the wound or surrounding tissue.

In a certain embodiment, the BBG of the present invention comprises boron. For example, the BBG of the present invention may comprise at least about 30 wt. %, at least about 35 wt. %, at least about 40 wt. %, at least about 45 wt. %, at least about 50 wt. %, at least about 55 wt. %, at least about 60 wt. %, at least about 65 wt. %, at least about 70 wt. %, at least about 75 wt. %, at least about 80 wt. %, or at least about 85 wt. % of boron. In further embodiments, at least a portion of the boron may be doped. For example, at least a portion of the boron may be doped with an element selected from the group consisting of Ca, Na, P, Cu, Zn, Ag, and combinations thereof. Without being bound by the theory, it is believed that embodiments wherein a portion of the boron is doped with another element result in improved biological performance by stimulating angiogenesis, antimicrobial properties, or other beneficial effects.

The hydrogel or hydrogel matrix of the present invention may comprise at least one component selected from the group consisting of alginate, gelatin, GelMa, cellulose, chitosan and combinations thereof. In further embodiments, the hydrogel or hydrogel matrix of the present invention may comprise one or more of living cells selected from the group consisting of epidermal keratinocytes, dermal fibroblasts, and mesenchymal stem cells. The living cells are capable of migration, proliferation to the top and/or bottom, and survival within the hydrogel, hydrogel matrix, or resulting 3D printed constructs or wound dressings.

In certain embodiments, the 3D printed constructs comprising the BBG and hydrogels are sterilized. In other embodiments, the bioactive borate glass (BBG) loaded hydrogel matrix is sterilized. In still further embodiments, the wound dressing is sterilized.

One particular aspect of the present invention is directed to a novel burn wound dressing product, comprising: a bioink in the form of paste made by adding up to 50 wt./v % bioactive borate glass (BBG) to a hydrogel matrix selected from the group consisting of alginate, gelatin, GelMa, cellulose, chitosan and combinations thereof. The viscosity of the BBG-hydrogel mixture may be adjusted by controlling the temperature, mixing speed, aging time, BBG/hydrogel ratio, and hydrogel compositions. To fabricate the wound dressing product, the BBG-hydrogel paste (i.e. a bioink) is placed in a syringe barrel for extrusion-based 3D printing. The hydrogel matrix is thought to be covalently cross-linked by the ions released from the BBG after mixing with the BBG. The hydrogel matrix may further comprise one or more types of living cells, for example, epidermal keratinocytes, dermal fibroblasts, or mesenchymal stem cells. The bioink and/or resulting wound dressing product is effective for the healing of burn wounds by providing a controlled ion release from the BBG, with the hydrogel serving as a continuous water source.

In addition to 3D printing technology, the novel BBG-hydrogel composite materials, bioinks, and BBG loaded hydrogel matrices can also be used to produce wound dressings using conventional manufacturing methods, such as molding and stamping.

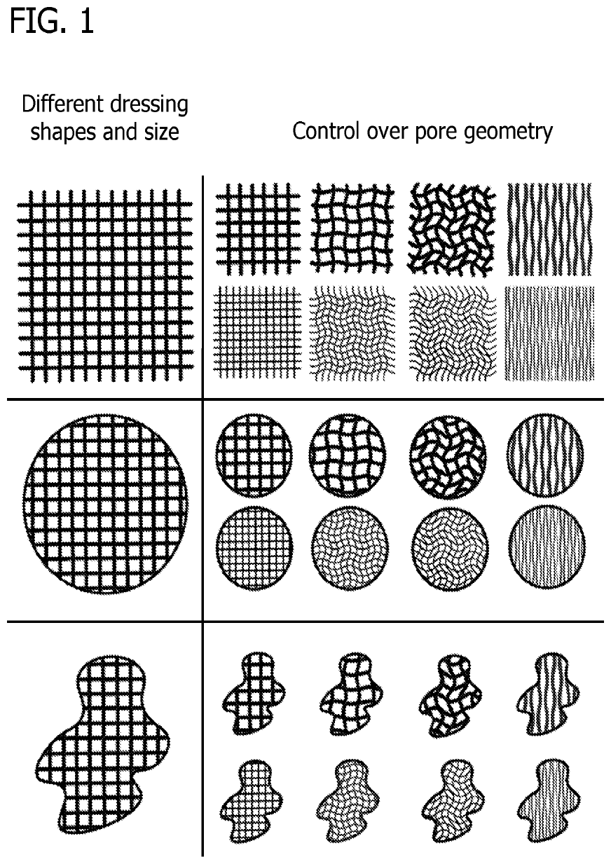

FIG. 1 provides an illustration of different dressing shapes, sizes, and pore geometries for 3D printing of porous BBG-hydrogel layers. The surface contact can be adjusted by altering the pore shape, size, and porosity to increase the cell-biomaterial interaction surface. The dressing shape and size can be tailored by the 3D printing technology to the wound geometry, if needed. For example, the advantages of using curved patterns over straight patterns include increased contact surface and more favorable anchors with curvatures for cell attachment.

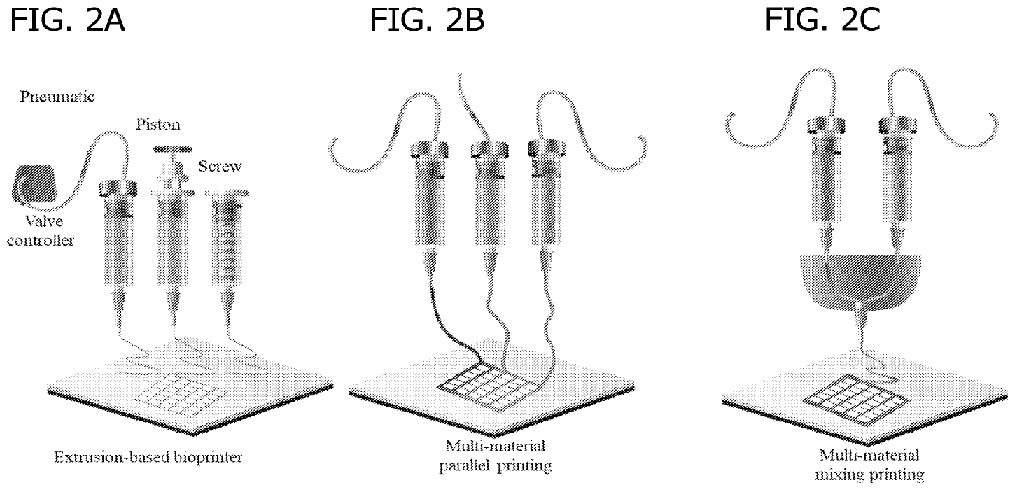

FIG. 2 illustrates an extrusion-based 3D printing technology apparatus. FIG. 2A shows three mechanisms of bioink extrusion: pneumatic pressure, electric drive with a piston, and electric drive with a screw (augur). FIG. 2B illustrates that it is possible to use multiple extruders (e.g. three extruders as shown) to deposit different bioinks for the production of wound dressings with multiple, inhomogeneous materials. FIG. 2C further illustrates that it is possible to have the multiple bioinks mixed and their mixing ratios continuously varied and controlled during the fabrication of the wound dressing. Regardless of the printing configuration, the wound dressings described herein can be printed at any suitable temperature, including room temperature.

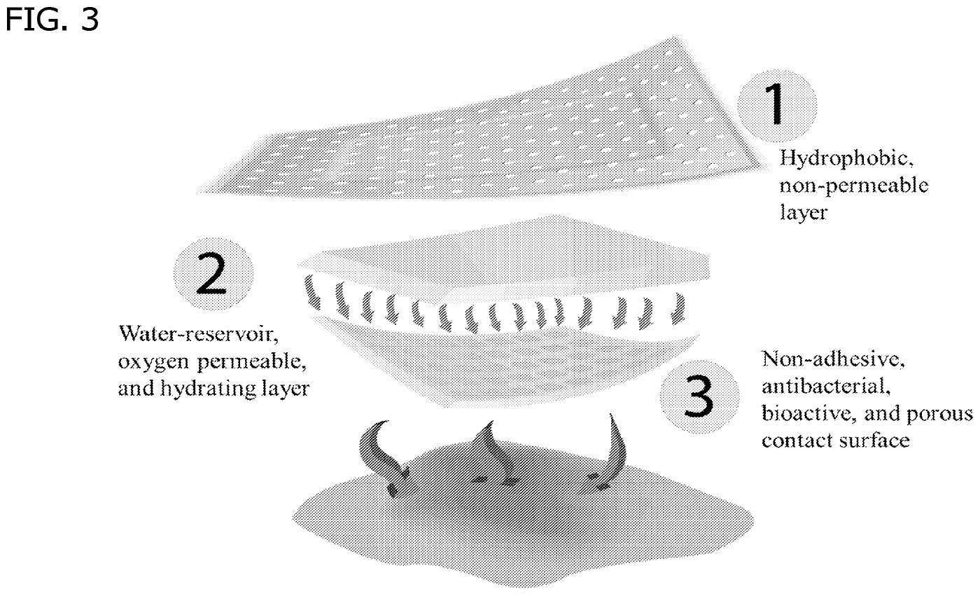

FIG. 3 shows a wound dressing, illustrating the flexibility and advantages of 3D printing, which can print a wound dressing product in three layers. The outermost layer protects the wound and underlying layers from the external environment and possible contamination as well as from water evaporation and loss. A middle layer is a non-porous hydrogel layer that maintains hydration at the optimum level. The contact (bottom) layer comprises BBG nanoparticles and a non-adhesive surface for non-traumatic dressing removal and increased wound-dressing interaction.

### EXAMPLES

Technical details regarding 3D printing of BBG-hydrogel bioinks to fabricate wound dressings, their mechanical and physical properties, and the results of in vitro and in vivo tests, are described below. All experiments were performed with at least 3 replications in order for the results to be statistically meaningful. Comparisons for statistically significant differences among the groups were performed using one-way ANOVA with significance set at p-value <0.05.

### Example 1

### BBG-Hydrogel Bioink Preparation

Various BBG-hydrogel compositions (i.e. bioinks) were prepared by mixing natural hydrogels such as gelatin, alginate, GelMa, cellulose, and chitosan with varying amounts of BBG, up to 50 w/v %, under controlled conditions including temperature, mixing speed, and aging time (the incubation time that BBG-hydrogel compositions were subjected to after mixing).

### 3D Printing

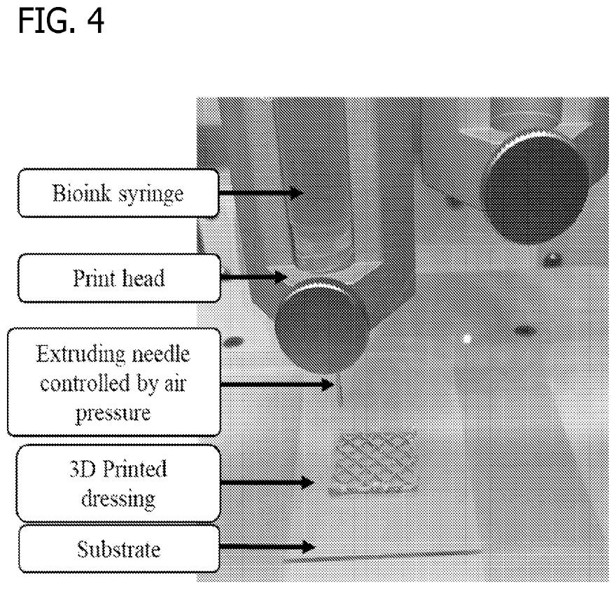

Each BBG-hydrogel composition (i.e. bioink) was loaded into a sterile syringe mounted on the print head of an extrusion-based 3D printer. The printing parameters, including nozzle diameter, extrusion pressure, paste temperature, and printing speed, were modified based on the viscosity of each hydrogel and the desired shape fidelity of the final printed construct. FIG. 4 shows a 3D printed BBG-hydrogel construct comprising 40 wt. % BBG and 60 wt. % hydrogel and the process equipment used to conduct the 3D printing. After printing, each 3D printed construct was placed in a Petri dish and exposed to a Ca-based salt solution for a period of time to create crosslinks between the hydrogel networks, where crosslinking was attributed to the ions released from the BBG.

### Rheology Test

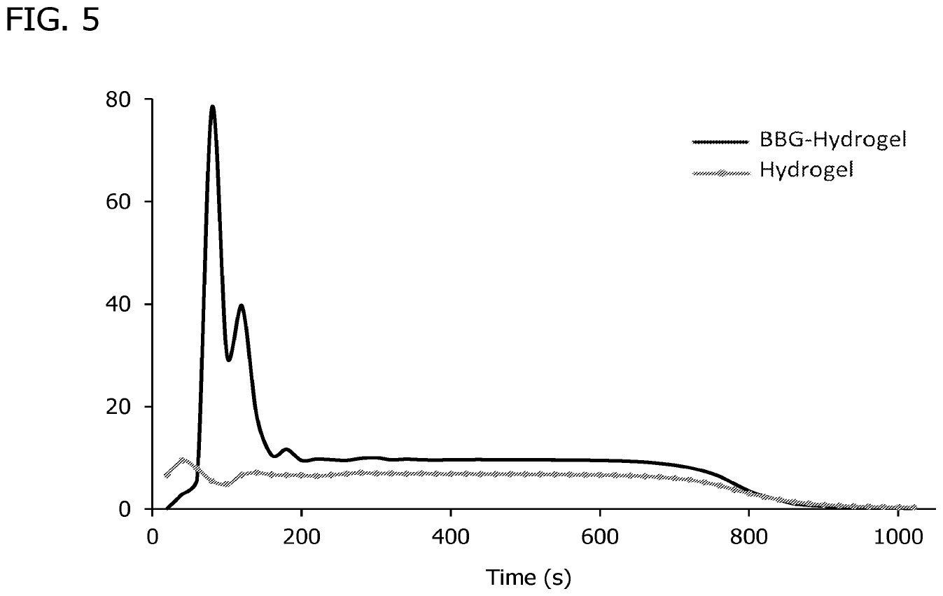

To control the extrusion-based 3D printing process, the rheological behavior of a hydrogel with and without BBG was measured. FIG. 5 plots G′/G″ of BBG-hydrogel vs. a hydrogel without BBG. G′ represents the elastic shear modulus and G″ represents the viscous shear modulus. The G′/G″ ratio corresponds to the relationship between the energy storage and loss, indicating how likely the printed material will be to return to its original shape when a force is applied to it. The BBG-hydrogel showed a higher G′/G″ ratio, which indicated that it had a higher potential to retain the shape compared to a hydrogel without BBG.

### Mechanical Test

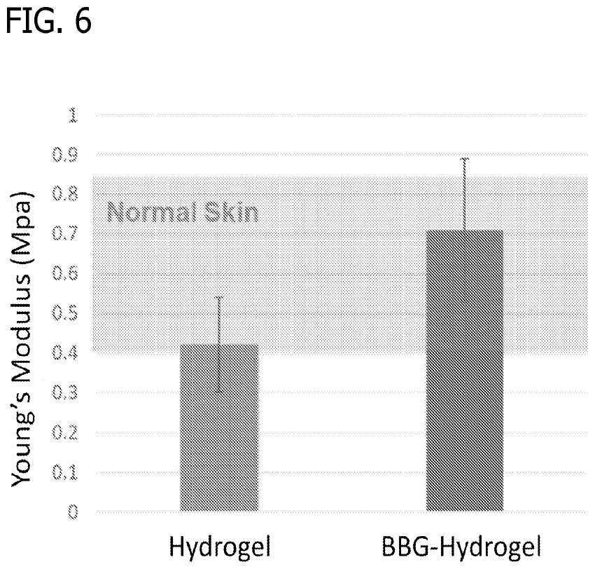

In accordance with the ASTM F2150-8 standard, the tensile strength and Young's modulus of the 3D printed BBG-hydrogel and hydrogel constructs were measured using an Instron 5969 Universal Testing System. The measurements were conducted on samples having dimensions of 40×10×2 mm 3, with the measurements taken in triplicate. FIG. 6 shows the Young's modulus of each of the two materials (BBG-hydrogel and hydrogel). The Young's modulus of both samples was in the same range as normal skin, with the hydrogel sample only barely falling within the range of normal skin. The BBG-hydrogel had a higher Young's modulus due to the crosslinking caused by ion release from the BBG to the hydrogel network. It is desirable for wound dressings to have a stiffness similar to that of normal skin to support body movement, non-adhesive coverage, and persistence on the wound site.

### Degradation Tests

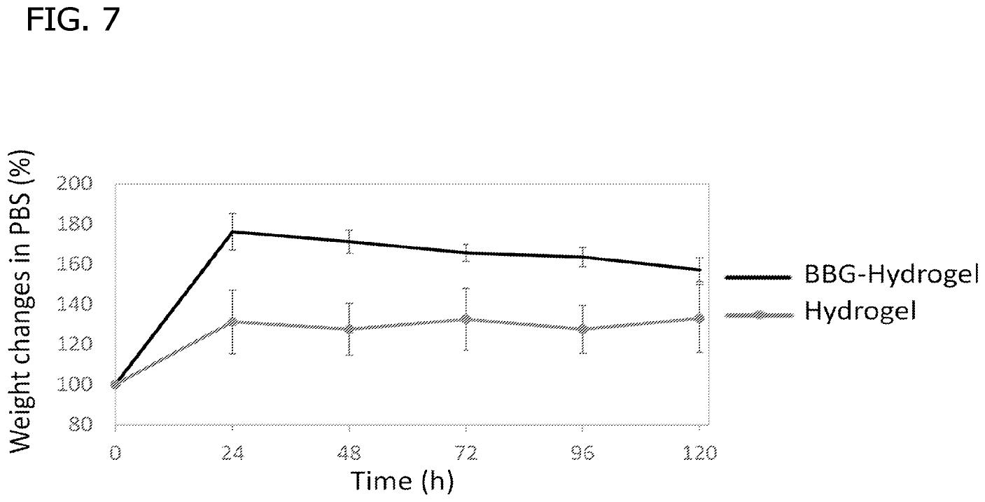

To understand and predict the further interactions between the wound and the dressing, the change in the weight of the 3D printed constructs made of BBG-hydrogel and hydrogel were recorded after soaking the constructs in a phosphate buffered saline (PBS) solution. The 3D printed samples were immersed in PBS at 32° C. and weighed every 24 hours. According to the data shown in FIG. 7, the 3D printed samples swelled (i.e. absorbing water within the hydrogel network) for both the BBG-hydrogel and hydrogel samples in the first 24 hours. The BBG-hydrogel sample had higher swelling after the first 24 hours, indicating a higher hydrophilicity in this sample due to the presence of ions in the BBG. After 24 hours, both samples showed weight loss attributable to the water molecules attack on the hydrogel network. The BBG-hydrogel sample showed a faster degradation rate after swelling, which indicates its usefulness for long-lasting water release from the sample. After 120 hours immersion in PBS, the weight of the BBG-hydrogel was still higher than the initial weight. The controlled water release and degradation is thought to be due to the formation of ionic bonds between the glass ions of the BBG and the hydrogel network. This allows for long-term moisture retention within the BBG-hydrogel network, which is highly desirable for burn wound healing.

### Hydration Tests

To evaluate the moisturizing activity of the 3D printed constructs, the total water content was measured using thermal analysis. Samples of BBG-hydrogel and hydrogel constructs were weighed and then placed in an oven at 250° C. for 10 minutes. The weight change was recorded based on the total water content within the sample. To understand and predict the hydration activity of the samples to burn wounds, two different absorbent foams composed of ethyl cellulose and polyurethane were used to represent superdehydrated and dry wounds, respectively. The 3D printed constructs were placed on the wound foam surfaces at 32° C., and hydration activity was measured by recording the weight change after 24 h at 32° C.

The total water content and donated water (hydration activity) were measured using the following equations:

$$Total water content ( \% ) = \frac{( {W}_{0} - {W}_{H} )}{{W}_{0}} \times 100 \qquad ( 1 )$$

$$Hydration activity ( \% ) = \frac{( {W}_{0} - {W}_{24} )}{{W}_{0} - {W}_{H}} \times 100 \qquad ( 2 )$$

where W₀ is the initial weight, W_{H} is the weight after heating at 250° C., and W₂₄ is the weight after placing on dry surfaces for 24 hours.

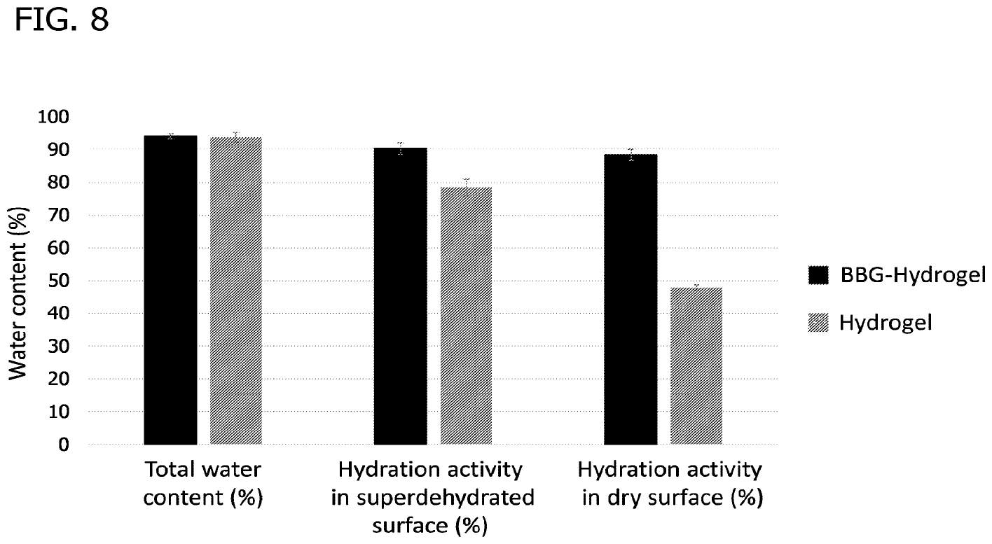

According to the results, presented in FIG. 8, there was no significant difference in the total water content between the BBG-hydrogel and hydrogel samples. The 3D printed BBG-hydrogel constructs donated more water than the 3D printed hydrogel constructs on both superdehydrated and dry surfaces, indicating that BBG-hydrogel has a higher moisturizing activity than the non-BBG hydrogel.

### Example 2

### Cell Viability Test

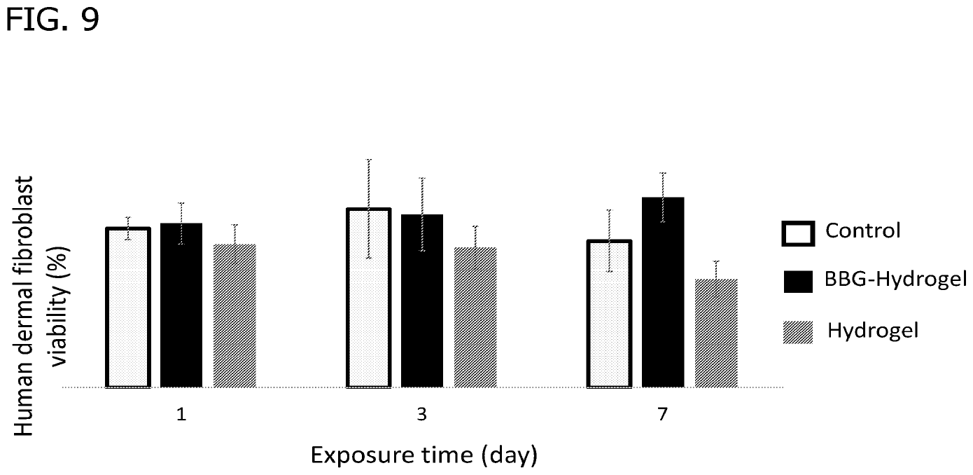

An MTT (3-(4,5-dimethylthiazol-2-yl)-2,5-diphenyltetrazolium bromide) assay was used to test and compare the viability and proliferation of skin cells in direct and indirect contact with a 3D printed hydrogel and BBG-hydrogel. Human primary dermal fibroblasts (HDF) were cultured in minimum essential media (MEM) fortified with 10% fetal bovine serum (FBS) and 1% pen/strep antibiotic at 37° C., in accordance with the ISO-10993 standard. 3D printed constructs were immersed in MEM for 1, 3, and 7 days. Indirect cell viability was assessed using an MTT assay and a colorimetric assay was used to assess the cell metabolic activity. Live cells react with MTT to form formazan, which can be measured by an ELISA reader as optical density. The chart set forth in FIG. 9 provides the measured MTT assay data. The data shows that the BBG-hydrogel and hydrogel samples along with control samples (i.e., HDF with no further treatment) had no significant difference after 1 day. At day 3, BBG-hydrogel and control samples had no significant difference, but both had higher cell viability compared to hydrogel samples. The BBG-hydrogel sample had significantly higher cell viability comparing to both the hydrogel and control samples after 7 days. This result indicates that ions released from BBG increased the cell growth and proliferation. From this data, it can be inferred that the release of ions from BBG in 3D printed BBG-hydrogel constructs increases the cell viability and bioactivity.

### Example 3

### In Vivo Wound Healing

The ability of the 3D printed skin substitutes for wound healing was evaluated by creating a circular full-thickness wound using a hot metal bar on the lumbar area of 36 Sprague Dawley rats in 6 groups, each group comprising 6 rats. The tested groups were as follows:
1) Control: wounds left with no treatment
2) BGG powder: wounds covered with BBG powder (particle size: 22 μm)
3) Non-printed hydrogel: wounds covered with non-printed hydrogel dressing
4) Non-printed BBG-hydrogel: wounds covered with non-printed BBG-hydrogel dressing
5) 3D printed hydrogel: wounds covered with 3D printed hydrogel dressing
6) 3D printed BBG-hydrogel: wounds covered with 3D printed BBG-hydrogel dressing

After shaving the back area of each rat, the skin was cleaned with iodine and then sterilized with alcohol swaps. The animals were anesthetized using inhaled isoflurane via a nose cone. The full-thickness defect was made by placing a 100° C. metal bar on the lumbar area of the rat for 10 seconds. After implementation, the wounds were disinfected by Dermoplast antiseptic spray. After applying the treatment, wounds were covered with petrolatum gauze, 3M bandage, and Elastikon tape. All animals were monitored daily for post-operative recovery, and the wounds were inspected under isoflurane anesthesia every week to record the wound size, re-epithelialization, and necrotic tissue formation. The necrotic tissue removal (i.e. debridement) was performed using sharp surgical tools. After 4 weeks of treatment, the experiment was terminated, and the animals were euthanized with a lethal dose of CO₂. Wound tissues were incised and fixed in formalin solution for 24 hours for further analysis.

Each week after the old dressing removal and prior to rebandaging, wounds were photographed to record the wound size, color, necrotic tissue formation, and traumatic removal. The wound size was quantified by tracing the wound border in each photograph using ImageJ software. The scale was set to a sterile disposable ruler in the wound picture, and wound closure was calculated as follows:

$$Wound closure ( \% ) = \frac{{A}_{0} - {A}_{t}}{{A}_{0}} \times 100 \qquad ( 3 )$$

where A₀ is the wound area immediately after wound implementation, and Aₜ is the wound area at time t (i.e., 1, 2, 3, and 4 weeks). Traumatic removal was evaluated by assessing the presence of traumatic laceration, bleeding, and redness in wound margins and surrounding tissues after the dressing removal.

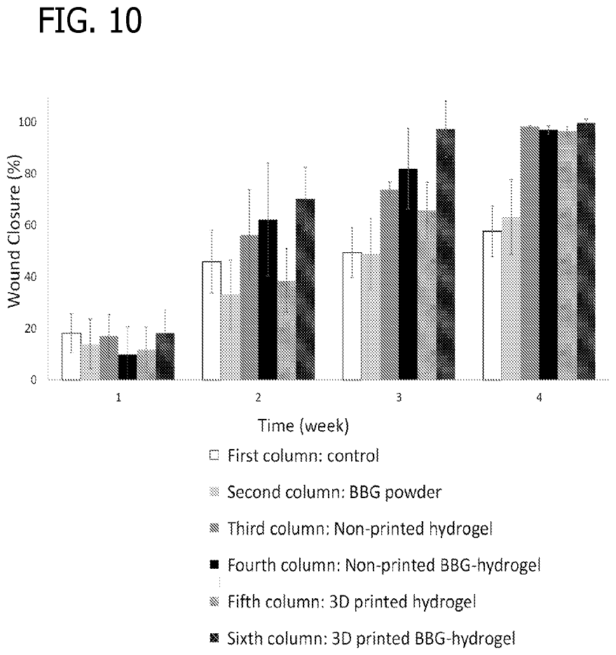

None of the tested treatment methods resulted in an infection. According to FIG. 10, the 3D printed BBG-hydrogel group showed the fastest wound healing with 100% wound closure after 4 weeks, followed by 3D printed hydrogel and non-printed samples. The control (i.e. petrolatum gauze) and BBG powder samples exhibited open wounds after 4 weeks, which was regarded as a failed wound treatment. The results of this experiment highlight the fact that BBG without a hydrogel is not effective on burn wound healing, due to the lack of a hydration source. The 3D printed dressings (with and without BBG) were non-adhesive with easy removal and no damage to the wound or surrounding tissue. The control, BBG powder, and non-printed hydrogels were adhesive to the wound surface with traumatic removal. The 3D printed and non-printed BBG-hydrogel dressings enabled autolytic debridement with no need for sharp instrument and surgical procedure to remove the necrotic tissue.

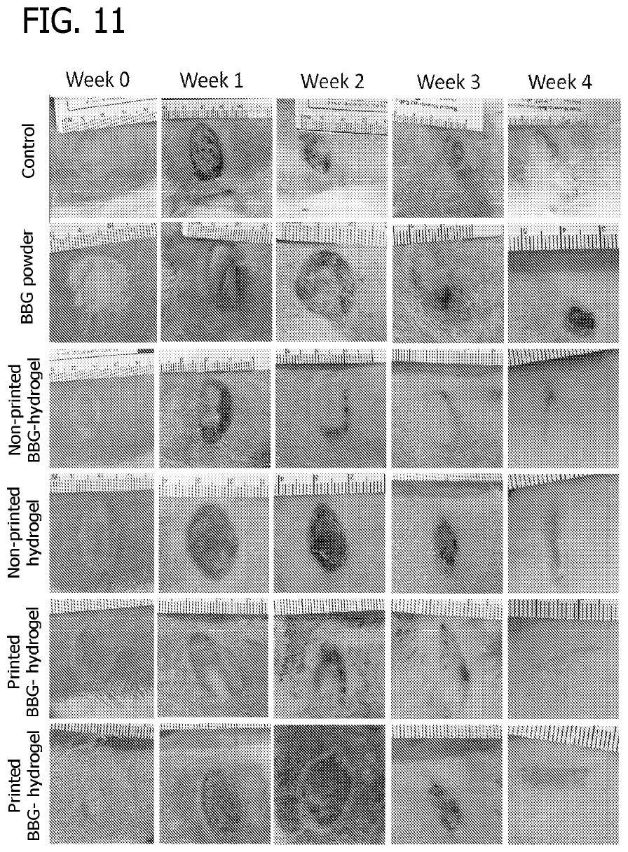

FIG. 11 shows the images of gross examination of the wounds to illustrate the effect of each treatment on epidermal growth, cutaneous regeneration, and wound appearance during the in vivo experiment. Re-epithelialization (i.e. formation of epidermis islands at the wound margin) was significantly higher with the 3D printed hydrogel, while wound closure took place faster with less scarring in the hydrogel-BBG samples. The plain BBG sample showed the highest amount of necrotic tissue and non-healing wound at the end of the experiment. With respect to the wound appearance, the 3D printed BBG-hydrogel dressing showed the lowest visibility in the wound margin, which indicates that this sample had the best effect of inhibiting further scar tissue formation. The combination of BBG and hydrogel clearly improved the appearance of regenerated skin with more homogenized re-epithelialization, while 3D printed samples showed faster wound healing than non-printed samples.

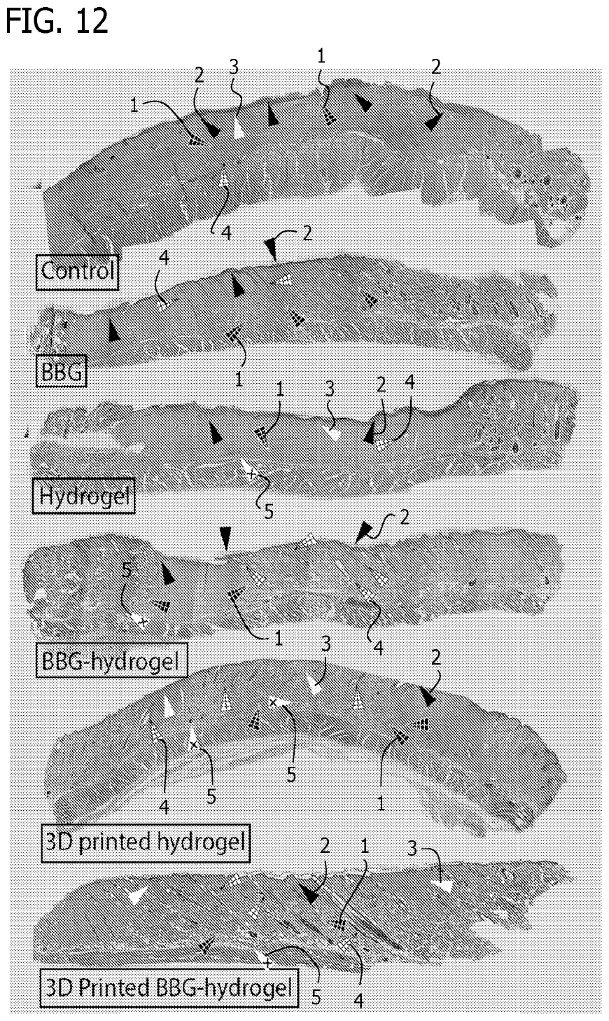

FIG. 12 shows H&E-stained slides for the 3D printed dressings, non-printed dressings, plain BBG powder, and the control group. FIG. 12 represents a 10× magnification. The control group (i.e. petrolatum gauze) and BBG powder showed the lowest epidermal regeneration (dark outmost layer) with relatively thicker hyperkeratosis (black arrowhead—reference number 1), as compared to the other four groups. The non-printed hydrogel and BBG-hydrogel showed relatively thicker hyperkeratosis compared to the 3D printed hydrogel and BBG-hydrogel with same compositions, which shows the positive effect of 3D printing fabrication on minimizing the hyperkeratosis. The dermal regeneration (white arrowhead—reference number 2) was approximately the same in all dressings, while the control group showed granulation tissue (dark-hatched arrowhead—reference number 3) instead of dermal regeneration. Granulation tissue refers to the chronically vascularized tissue with persisted inflammation that is mainly composed of red to pink granular tissue. Formation of granulation tissue after 28 days is a major indication of immature wound healing and failed treatment. The control group showed the highest granulation tissue followed by BBG powder. Both non-printed and 3D printed dressings with and without BBG had lower granulation tissue. The 3D printed hydrogel and BBG-hydrogel dressings showed a distinctive formation of hair follicles due to the continuous hydration and non-stick surface with aligned pores. The number of hair follicles (light-hatched arrowheads—reference number 4) was significantly higher in the 3D printed BBG-hydrogel than the other groups. Also, the hair follicles in the 3D printed BBG-hydrogel group showed significantly higher growth from the dermal layer to the epidermal layer and beyond, followed by the non-printed BBG-hydrogel and 3D printed hydrogel. In the non-printed hydrogel dressing and the control group, hair follicles were still in the dermal layer, which implies the growth and development of hair follicles began after 4 weeks. Several sweat glands (x-containing arrowheads—reference number 5) were regenerated in all groups, with slightly higher wound regeneration in the 3D printed dressings. Thus, it can be concluded that the presence of BBG can improve the regeneration of hair follicles, which is amplified by 3D printing due to the continuous hydration and non-stick surface with aligned pores. In both 3D printed and non-printed BBG-hydrogel groups, hair follicles grew from the dermal layer to the epidermal layer, which means the growth and development of hair follicles in these groups is faster than the other groups. These findings indicate the positive effect of BBG on regeneration of hair follicles during burn wound healing.

Overall, the in vivo results appeared to indicate that the positive effect of 3D printed dressings on burn wound healing comes from the increased degradation, hydration, and surface contact, and the combination of BBG and hydrogel exhibits improved results, with BBG providing vital biological effects and the hydrogel serving as a water reservoir for burn wound healing.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions, methods and processes without departing from the scope of the invention, it is intended that all matter contained in the above description and shown in the accompanying drawings shall be interpreted as illustrative and not in a limiting sense.

1. A wound dressing comprising a hydrogel matrix and up to 50 w/v % of a bioactive borate glass (BBG) comprising boron.

2. The wound dressing of claim 1, at least a portion of the boron is doped with an element selected from the group consisting of Ca, Na, P, Cu, Zn, Ag, and combinations thereof.

3. The wound dressing of claim 1, wherein the BBG comprises at least about 30 wt. %, at least about 35 wt. %, at least about 40 wt. %, at least about 45 wt. %, at least about 50 wt. %, at least about 55 wt. %, at least about 60 wt. %, at least about 65 wt. %, at least about 70 wt. %, at least about 75 wt. %, at least about 80 wt. %, or at least about 85 wt. % of boron.

4. The wound dressing of claim 1, wherein the hydrogel comprises at least one component selected from the group consisting of alginate, gelatin, GelMa, cellulose, chitosan and combinations thereof.

5. The wound dressing of claim 1, wherein the hydrogel matrix further comprises one or more of living cells selected from the group consisting of epidermal keratinocytes, dermal fibroblasts, and mesenchymal stem cells.

6. The wound dressing of claim 1, wherein the dressing is non-adhesive to a wound, a burn, or a site of bleeding.

7. The wound dressing of claim 1, wherein the dressing is capable of autolytic debridement when applied to a wound, a burn, or a site of bleeding and/or is capable of reducing or inhibiting scar tissue formation when applied to a wound, a burn, or a site of bleeding.

8. (canceled)

9. A process for preparing a bioactive borate glass (BBG) loaded hydrogel matrix, the process comprising:
providing a paste comprising hydrogels and BBG;
additive manufacturing of the paste comprising hydrogels and BBG using extrusion-based 3D printing to form 3D printed constructs comprising the BBG and hydrogels; and
crosslinking the 3D printed constructs to form a hydrogel matrix comprising up to about 50 w/v % BBG.

10. The process of claim 9, further comprising sterilizing the 3D printed constructs.

11. The process of claim 9, wherein the hydrogel comprises at least one component selected from the group consisting of alginate, gelatin, GelMa, cellulose, chitosan, and mixtures thereof.

12. The process of claim 9, wherein the paste comprising hydrogels and BBG is 3D printed in combination with one or more of living cells selected from the group consisting of epidermal keratinocytes, dermal fibroblasts, or mesenchymal stem cells.

13. The process of claim 9, where the 3D printed constructs have different shapes, sizes, and/or pore geometries.

14. The process of claim 9, where the 3D printed constructs are formed in situ.

15. The process of claim 9, where the 3D printed constructs are not formed in situ.

16. The process of claim 9, where the 3D printed constructs are formed with multiple materials that may vary between layers and within each layer as specified.

17. The process of claim 9, wherein human dermal fibroblasts and/or keratinocytes are capable of migration and survival within the 3D printed constructs.

18. The process of claim 9, wherein human dermal fibroblasts and/or keratinocytes are capable of migration or proliferation to the top and/or bottom of the 3D printed constructs.

19. A method of treating wounds, burns, and/or controlling bleeding in a subject in need thereof, the method comprising applying a wound dressing of claim 1 to a wound, a burn, or a site of bleeding of the subject.

20. The method of claim 19, wherein the wound dressing is capable of commercially acceptable moisture retention for up to 7 days after application to the wound, burn, or site of bleeding.

21. The method of claim 19, wherein the wound dressing exhibits antibacterial activity for up to 7 days after application to the wound, burn, or site of bleeding.
